(19) United States
(12) Patent Application Publication
(10) Pub. No.: US 2026/0347295 A1
(43) Pub. Date: Nov. 5, 2026

(54) SYNTHESIS OF METHANOL

(71) Applicant: L'Air Liquide, Société Anonyme pour I'Etude et I'Exploitation des Procédés Georges Claude, Paris (FR)

(72) Inventors: Tobias OELMANN, Bad Vilbel (DE); Michael STROZYK, Offenbach (DE); Vincent PENA, Frankfurt (DE)

(21) Appl. No.: 19/486,676

(22) PCT Filed: May 24, 2024
(86) PCT No.: PCT/EP2024/064405

§ 371 Date: Nov. 21, 2025

(30) Foreign Application Priority Data
Jun. 7, 2023 (EP) 23177951.3

(51) Int. Cl. C07C 29/152; C07C 29/151; C07C 29/76; C07C 31/04

(52) U.S. Cl. CPC C07C 29/152; C07C 29/1516; C07C 29/76; C07C 31/04

(57) ABSTRACT

A process for methanol synthesis using a pipe conduit system with integrated raw gas source, methanol synthesis reactor and a separation means, wherein a feed gas stream including reactants for methanol synthesis is supplied from the raw gas source to the methanol synthesis reactor at a volume flow rate, wherein methanol is produced in the methanol synthesis reactor, wherein the methanol produced is separated from an outlet stream of the methanol synthesis reactor with the separation means, wherein the pipe conduit system further has an auxiliary volume integrated into it such that a gas-accessible volume of the pipe conduit system depends on a fill level of a liquid in the auxiliary volume and wherein the fill level of the liquid in the auxiliary volume is increased at least in a case of a reduction in the volume flow rate of the feed gas stream.

### CROSS REFERENCE TO RELATED APPLICATIONS

This application is a 371 of International Application No. PCT/EP2024/064405, filed May 24, 2024, which claims priority to European Patent Application No. 23177951.3, filed Jun. 7, 2023, the entire contents of which are incorporated herein by reference.

### BACKGROUND

The present invention relates to a process and an apparatus for synthesis of methanol.

Methanol may be produced with a methanol plant which comprises a so-called synthesis loop. This is a pipe conduit system into which a raw gas source, a methanol synthesis reactor, a heat exchanger and a separator are typically integrated.

One of the reactants of the methanol synthesis is hydrogen. In order to achieve a methanol synthesis that is as environmentally friendly as possible it is desirable to use hydrogen from a water electrolysis operated with renewable energy. However, renewable energies fluctuate and so the availability of hydrogen from a corresponding source also fluctuates. Fluctuations in the feed gas flow cause pressure fluctuations in the synthesis loop during methanol synthesis. However, a rapid pressure drop in the synthesis loop can cause damage to the installations used, especially to a catalyst used in methanol synthesis. It is therefore desirable to keep the pressure in the synthesis loop as stable as possible.

Conventional methanol plants are therefore not typically operated in alternating load operation. Operation under partial load is typically defined as operation at at least about 70% of plant capacity. In a conventional methanol plant partial load operation is typically selected only in the case of reduced gas availability, for example in winter, which extends over longer periods in the order of weeks and months and can be planned in advance. A change to a steady-state partial load operation is thus carried out over hours or days with very slow process adaptations to protect the equipment, in particular the catalyst.

Especially in small plants, especially in CO₂-based syntheses with hydrogen from an electrolysis, alternating load operation in a range between 20% and 100% is desirable in order to take account of the availability of renewable energy for example. Conventional methanol plants can realize this alternating load operation only with a disproportionate level of stress on the equipment, if at all.

### SUMMARY

It is an object of the present invention to provide an option for producing methanol with a particularly low level of stress on the employed equipment even in the case of fluctuating supply of the reactants.

These objects are achieved with the process and the apparatus according to the independent claims. Further advantageous embodiments are specified in the dependent claims. The features presented in the claims and in the description may be combined with one another in any technologically advantageous way.

The present invention provides a process for synthesis of methanol using a pipe conduit system into which a raw gas source, a methanol synthesis reactor and a separation means are integrated, wherein a feed gas stream comprising reactants for methanol synthesis is supplied from the raw gas source to the methanol synthesis reactor at a volume flow rate, wherein methanol is produced with the reactants in the methanol synthesis reactor, wherein the methanol produced is separated from an outlet stream of the methanol synthesis reactor with the separation means, wherein the pipe conduit system further has an auxiliary volume integrated into it such that a gas-accessible volume of the pipe conduit system depends on a fill level of a liquid in the auxiliary volume and wherein the fill level of the liquid in the auxiliary volume is increased at least in a case of a reduction in the volume flow rate of the feed gas stream.

The described process makes it possible to produce methanol using a pipe conduit system. The pipe conduit system is part of an assembly for synthesis of the methanol. At least a raw gas source, a methanol synthesis reactor and a separation means are integrated into the pipe conduit system. Further components can also be integrated into the pipe conduit system. These may be configured for the synthesis of methanol similarly to a conventional assembly. If such further components are not described herein these are immaterial at least in terms of the particularities of the described process.

The way in which the pipe conduit system is configured is also immaterial. In the simplest case the pipe conduit system comprises a first conduit by which an outlet of the raw gas source is connected to an inlet of the methanol synthesis reactor and a second conduit by which an outlet of the methanol synthesis reactor is connected to an inlet of the separation means. It is preferable when the pipe conduit system further comprises a third conduit by which a gas outlet of the separation means is connected to the first conduit. Gas in the pipe conduit system may be recycled i.e. returned to the methanol synthesis reactor via the third conduit. If the pipe conduit system comprises the third conduit or an equivalent option for recirculation the pipe conduit system may also be described as a loop, in particular as a synthesis loop. In addition to the described two or three conduits the pipe conduit system may comprise various further conduits, branches and interconnections.

The raw gas source may be a source for a raw gas which already comprises a mixture of all reactants of the methanol synthesis. However, the raw gas source may also comprise a plurality of individual sources, each of which only contributes one of the reactants. The corresponding individual streams may be combined into the feed gas stream. Where this is done is immaterial to the described process.

In the case of the described process a feed gas stream comprising reactants for methanol synthesis is supplied from the raw gas source to the methanol synthesis reactor. This is done at a volume flow rate which is more particularly described below. The reactants for the methanol synthesis may be in particular hydrogen on the one hand and carbon monoxide and/or carbon dioxide on the other hand. The hydrogen preferably derives at least partially from a hydrogen electrolysis operated with renewable energies.

Methanol is produced with the reactants in the methanol synthesis reactor. This may be done in a manner known per se, in particular using a catalyst in the methanol synthesis reactor. The methanol synthesis reactor may discharge an outlet stream which contains not only the methanol but also further substances, in particular H₂O formed during the methanol synthesis and the reactants to the extent that these have not reacted in the methanol synthesis reactor. The methanol produced is separated from the outlet stream of the methanol synthesis reactor with the separation means. This may also be done in a way that is known per se. The separation means may be a liquid separation means. The separation means may especially be adapted for separating a gas phase and a liquid phase. In this case the methanol may be separated as a liquid. The outlet stream is commonly known as “raw methanol” although the outlet stream may also contain other substances in addition to the methanol.

In contrast to conventional processes for methanol synthesis, in the described process the volume of the pipe conduit system is deliberately altered. This takes advantage of the general principle that in the case of a gas at otherwise unchanged conditions a reduction in the available volume is accompanied by a pressure increase and vice versa. Changing the volume of the pipe conduit system accordingly makes it possible to influence the pressure in the pipe conduit system. In the described process this is utilized to be able to react to fluctuations in the raw gas stream. This is especially advantageous when the raw gas is produced at least partially with renewable energies, as may be the case in particular in the case of hydrogen as a reactant for methanol synthesis. If insufficient renewable energy is available it is not possible to produce sufficient hydrogen for full-load operation of the methanol synthesis. The feed gas stream is supplied to the methanol synthesis reactor with a reduced volume flow rate. This would in principle result in a reduction of the pressure in the pipe conduit system which in turn can lead to damage to components, in particular at the catalyst.

The described process can reduce or even completely prevent such a pressure reduction. To this end at least in a case of a reduction in the volume flow rate of the feed gas stream the gas-accessible volume of the pipe conduit system is reduced. This has the result that despite the reduction in the volume flow rate of the feed gas stream the pressure in the pipe conduit system is kept constant or falls only a little.

The gas-accessible volume of the pipe conduit system is altered with the aid of an auxiliary volume. The auxiliary volume is integrated into the pipe conduit system such that the gas-accessible volume of the pipe conduit system depends on a fill level of a liquid in the auxiliary volume. The auxiliary volume is part of the pipe conduit system. It may be configured in a conduit or in a component integrated into the pipe conduit system. The auxiliary volume is a space within the pipe conduit system which is not closed on all sides and is accordingly fluidically connected to the rest of the pipe conduit system. It is not necessary for the auxiliary volume to be formed by a specific component or otherwise to be distinguishable from the rest of the pipe conduit system. It is possible that in a specific pipe conduit system an auxiliary volume may be identified as of two or more different types and/or at two or more different locations. It is sufficient that liquid can collect in the auxiliary volume. The auxiliary volume may be formed in a dip in a pipe conduit for example. The precise configuration of the auxiliary volume is immaterial to the mode of operation of the process. Rather, the advantages described herein are achieved as a result of the auxiliary volume being used as described to adjust the gas-accessible volume of the pipe conduit system via the fill level of the liquid in the auxiliary volume.

The gas-accessible volume of the pipe conduit system is especially made up of the volumes of the conduits of the pipe conduit system and of the volumes of the components integrated into the pipe conduit system. For example, a gas-accessible portion of the methanol synthesis reactor contributes to the gas-accessible volume of the pipe conduit system. It can therefore be left undetermined where a boundary between a methanol synthesis reactor and a conduit connected thereto is drawn.

Provided the auxiliary volume is not filled with liquid the auxiliary volume likewise contributes to the gas-accessible volume of the pipe conduit system. The part of the auxiliary volume that is filled with the liquid is not gas-accessible on account of the liquid. The gas-accessible portion of the auxiliary volume can therefore be varied via the amount of the liquid in the auxiliary volume. This makes it possible to influence the gas-accessible volume of the pipe conduit system via a fill level of the liquid in the auxiliary volume. In the described process the fill level of the liquid in the auxiliary volume is therefore increased at least in a case of a reduction in the volume flow rate of the feed gas stream. Changing the fill level of the liquid in the auxiliary volume thus makes it possible to react to a drop in the volume flow rate of the feed gas stream.

The described process is especially suitable for reacting to rapid fluctuations in the feed gas stream. It is also possible to react to slower fluctuations by other means. In order to achieve an advantage relative to a conventional process it is therefore sufficient for the fill level of the liquid in the auxiliary volume to be increased at least in a case of a reduction in the volume flow rate of the feed gas stream. The behaviour of the fill level in the case of an increase in the volume flow rate of the feed gas stream is generally immaterial. The fill level of the liquid in the auxiliary volume is also immaterial in the case of a constant feed gas stream. It is sufficient if the fill level is increased for a short time at least in a case of a reduction in the volume flow rate of the feed gas stream to effect short-term compensation of the effects of reducing the volume flow rate of the feed gas stream. This is because the pressure in the pipe conduit system can also be influenced in other ways in addition to fill level control. All options that are also employed in a conventional assembly for synthesis of methanol are in particular contemplated in this regard. For example excessive pressure may be released via an overpressure valve which can react relatively quickly. It can also generally be assumed that in the production of hydrogen by electrolysis for example a rapid decrease in production is more likely to occur than a rapid increase in production. The risk of excessively high pressure is thus qualitatively distinct from the risk of excessively low pressure.

It is nevertheless preferable when the fill level is reduced at least in a case of an increase in the volume flow rate of the feed gas stream. In this case the change in the fill level helps to stabilize the pressure in the pipe conduit system. However, against the backdrop of the foregoing it is also possible that the fill level of the liquid in the auxiliary volume is initially increased in a case of a reduction in the volume flow rate of the feed gas stream and subsequently reduced again independently of the volume flow rate of the feed gas stream, for example to a value prevailing prior to the increase. It is immaterial when this reduction is effected.

It is also unnecessary for the fill level of the liquid in the auxiliary volume to be increased every time the volume flow rate of the feed gas stream falls. It is sufficient when the fill level of the liquid in the auxiliary volume is increased at least in a case of a reduction in the volume flow rate of the feed gas stream, i.e. at least once during performance of the process. Especially in the case of a slow reduction in the volume flow rate of the feed gas stream it is unnecessary to react by increasing the fill level of the liquid in the auxiliary volume. However, it is preferable when the fill level of the liquid in the auxiliary volume is increased every time the volume flow rate of the feed gas stream falls faster than a pressure change threshold value and/or is increased every time the volume flow rate of the feed gas stream falls to a value below a pressure threshold value.

The liquid in the auxiliary volume can also be referred to as auxiliary liquid on account of its function. However, it is not necessary for the auxiliary liquid to be used only for this function. The auxiliary liquid may especially also be a liquid which is in any case present in the pipe conduit system. Avoidance of the term auxiliary liquid is intended to clarify that the auxiliary liquid need not be a separate liquid. The auxiliary volume may also be referred to as a buffer volume. The liquid in the auxiliary volume may accordingly also be referred to as a buffer liquid.

In a preferred embodiment of the process the liquid comprises methanol.

Use of methanol as the liquid has the advantage that no foreign substance need be introduced into the pipe conduit system. The liquid is preferably pure methanol. This does not preclude the methanol containing unavoidable impurities. It is alternatively preferable when the liquid is a methanol-water mixture also referred to as “raw methanol” or “stabilized methanol” with small amounts of impurities such as are produced in methanol synthesis.

The liquid may also contain other substances alternatively or in addition to methanol. What is material to the essential mode of operation of the described process is merely that the liquid displaces gas and can thus influence the gas-accessible volume of the pipe conduit system. However, this is generally the case for liquids.

In a further preferred embodiment of the process the liquid comprises methanol produced in the methanol synthesis reactor. That the liquid comprises methanol is to be understood as meaning that the liquid is not necessarily pure methanol but rather can especially also be a mixture of methanol and for example water which may also contain unavoidable byproducts.

Use of methanol from the methanol synthesis reactor as the liquid has the advantage that no liquid need be supplied from an external source. The gas-accessible volume of the pipe conduit system may be influenced by merely altering the fill level of the methanol in any case present in the pipe conduit system in the auxiliary volume. This may be achieved via a pump for example. An external storage means for the liquid is unnecessary in this case.

In a further preferred embodiment of the process the fill level of the liquid in the auxiliary volume is increased at least in a case of a reduction in the volume flow rate of the feed gas stream by introducing the liquid into the auxiliary volume at least partially from an external storage means.

In contrast to the above-described embodiment, in this embodiment an external storage means is used. The external storage means is preferably a tank. The liquid in the external storage means may be formed by methanol and/or one or more other substances. The fluid in the external storage means may in particular be raw methanol, stabilized methanol, off-spec methanol or pure methanol.

The liquid in the auxiliary volume may be formed by liquid from the external storage means and/or by other liquid, in particular methanol from the methanol synthesis reactor. Also conceivable is the combination of embodiments according to which the liquid comprises methanol produced in the methanol synthesis reactor and the fill level of the liquid in the auxiliary volume is increased at least in a case of a reduction in the volume flow rate of the feed gas stream by introducing the liquid into the auxiliary volume partially from an external storage means. In the case of this combination it is particularly preferable when the liquid in the external storage means is methanol.

In a further preferred embodiment of the process the methanol synthesis reactor produces methanol at a production rate of up to 5000 tonnes per day.

The greater the auxiliary volume compared to the gas-accessible volume of the rest of the pipe conduit system, the more the total gas-accessible volume of the pipe conduit system can be influenced via a fill level alteration of the liquid in the auxiliary volume. The greater this mode of influencing, the more the described process can react to a variation in the feed gas stream and the greater the advantages of the described process. It is therefore desirable that the auxiliary volume be as large as possible relative to the gas-accessible volume of the rest of the pipe conduit system. For practical reasons the implementation of the auxiliary volume is generally increasingly difficult, the larger the auxiliary volume is intended to be. The described process is therefore particularly suitable for small to medium-sized assemblies for methanol synthesis. In the present embodiment this is quantified via the production rate of the methanol. If the methanol synthesis reactor produces methanol at a production rate of for example 1000 tonnes per day, the assembly may be described as a medium-sized assembly.

For very small assemblies having a production rate of up to 10 tonnes per day, the auxiliary volume may account for several times the gas-accessible volume of the rest of the pipe conduit system at relatively low cost and space requirements. Thus very small assemblies may be operated in a particularly flexible manner using the described process.

For small assemblies having a production rate between 100 and 1000 tonnes per day, the auxiliary volume may correspond to the gas-accessible volume of the rest of the pipe conduit system at acceptable cost. The described process can thus advantageously be employed with the aim of a particularly flexible operation in the case of small assemblies.

For medium-sized plants having a production rate of between 1000 and 5000 tonnes per day, an auxiliary volume of the same order of magnitude as the gas-accessible volume of the rest of the pipe conduit system is possible but represents a relevant space demand so that implementation of the presented measure may in this case be associated with additional challenges.

Alternatively to the described embodiment the process may also be employed in larger plants where the potentially greater cost of realizing the auxiliary volume is accepted.

A further aspect of the invention provides an assembly for synthesis of methanol. The assembly comprises a pipe conduit system and a raw gas source, a methanol synthesis reactor and a separation means which are integrated into the pipe conduit system, wherein the raw gas source, the methanol synthesis reactor and the separation means are connected to one another via the pipe conduit system, wherein the assembly further comprises an auxiliary volume which is integrated into the pipe conduit system in such a way that a gas-accessible volume of the pipe conduit system depends on a fill level of a liquid in the auxiliary volume and wherein the assembly further comprises a fill level control means which is adapted for increasing the fill level of the liquid in the auxiliary volume at least in a case of a reduction in a volume flow rate of a feed gas stream supplied to the methanol synthesis reactor from the raw gas source via the pipe conduit system.

The advantages and features of the process are applicable and transferable to the assembly and vice versa. The assembly is preferably adapted for operation according to the process. The process is preferably performed with the assembly.

As described in connection with the process a raw gas source, a methanol synthesis reactor and a separation means are integrated into the pipe conduit system. The raw gas source, the methanol synthesis reactor and the separation means are connected to one another via the pipe conduit system. The fact that the assembly is adapted for synthesis of methanol implies that the recited elements are connected to one another via the pipe conduit system in such a way that methanol can be produced in the methanol synthesis reactor.

The raw gas source is thus connected to the methanol synthesis reactor in such a way that a feed gas stream comprising reactants for methanol synthesis is suppliable from the raw gas source to the methanol synthesis reactor at a volume flow rate. The methanol synthesis reactor is adapted for producing methanol with the reactants. The methanol synthesis reactor is connected to the separation means in such a way that the methanol produced is separable from an outlet stream of the methanol synthesis reactor with the separation means.

The fill level control means is adapted for adjusting the fill level of the liquid in the auxiliary volume as described for the process. The fill level control means comprises all elements required for this purpose. The fill level control means may comprise for example a control means which via an input signal obtains information about the volume flow rate of the feed gas stream and via an output signal controls a pump such that the fill level of the liquid in the auxiliary volume is adjusted as described. The input signal may derive from a flow sensor which is likewise part of the fill level control means. Alternatively or in addition the fill level control means may for example also be connected to a coordinate control unit or be integrated into a superordinate control unit, wherein the information about the volume flow rate of the feed gas stream is in any case available in the coordinate control unit/the superordinate control unit. The pump too may be part of the fill level control means. Alternatively, the pump may also be used for other purposes. The pump preferably has an output pressure in the range from 50 to 150 bar, in particular in the range from 90 to 100 bar.

In a preferred embodiment of the assembly the auxiliary volume is configured in a separator together with the separation means.

In a conventional assembly for synthesis of methanol the methanol is separated from the outlet stream of the methanol synthesis reactor with a separator. This separator is integrated into the pipe conduit system as a separate component. However, in the present embodiment of the described process the auxiliary volume too is configured in the separator. This is why the term separation means is used here. The separation means comprises the elements used for separating the methanol from the outlet stream of the methanol synthesis reactor. In a conventional assembly for synthesis of methanol the terms separation means and separator may be used synonymously. However, in the present embodiment of the described process the separator is the superordinate unit which comprises the separation means and the auxiliary volume.

The underside of a conventionally used separator also has a region in which liquid can collect. The separator used in the present embodiment of the described process can therefore even correspond to a conventional separator in terms of its construction. However, in a conventional assembly the separator is not combined with the fill level control means described herein for the purpose of influencing the pressure in the pipe conduit system. In the simplest case the described assembly thus differs from a conventional assembly merely in terms of the configuration of the control means. However, the advantages described herein can be achieved to a particularly great extent when the employed assembly is modified with a view to fill level control. In particular, the advantages described herein are achievable particularly when the auxiliary volume is particularly large. In the case of a conventional separator there is no comparable motivation to provide a largest possible auxiliary volume in the separator.

In a further preferred embodiment of the assembly a cross-sectional area of the separator in the region of the auxiliary volume is at least 50% larger, preferably twice as large, as in the region of the separation means.

In this embodiment the separator is modified with a view to fill level control. To this end the region of the separator used for the auxiliary volume is particularly large. The separator may therefore be described as bulbous.

Alternatively or in addition to a particularly large cross section in the region of the auxiliary volume a particularly large auxiliary volume may also be achieved by a particularly large axial extent of the separator. However, the particularly large cross section in the region of the auxiliary volume has the advantage that even a vertical arrangement of the separator does not require a particularly great pumping power to introduce the liquid into the auxiliary volume.

In a further preferred embodiment of the assembly the auxiliary volume is arranged below the separation means.

Even a conventional separator generally provides a region in which the separated liquid methanol can collect below the separation means. The described assembly in the present embodiment is thus achievable without great retrofitting measures. It is even conceivable to use a conventional separator without modification. However, the finding described herein is that of using the lower region of the separator as the auxiliary volume and combining it with the described fill level control means.

The liquid may especially be raw methanol produced with the methanol synthesis reactor which has been separated by the separation means in an upper portion of a separator and has been separated into the auxiliary volume arranged below the separation means and has run or dripped downwards into the auxiliary volume.

Arranging the auxiliary volume below the separation means generally has the advantage that the separation means is thus arranged downstream of the auxiliary volume. Even if a portion of the liquid from the auxiliary volume were to be entrained by a gas stream in the pipe conduit system this portion of the liquid would immediately be re-separated from the gas stream by the separation means. There is thus no risk of droplets being entrained with the gas stream. The latter is especially relevant for the preferred case where the assembly is configured such that the outlet stream of the methanol synthesis reactor may be recirculated. In that case the entrained droplets could pass into a recycle conduit. This can be prevented by the separation means.

In a further preferred embodiment of the assembly the auxiliary volume is configured in a vessel which is integrated into the pipe conduit system between the methanol synthesis reactor and the separation means.

As an alternative to the above-described embodiments in which the auxiliary volume is configured in the separator, in this embodiment the auxiliary volume is configured outside the separator.

In a further preferred embodiment of the assembly the vessel is configured as a knock-out drum.

In this embodiment the auxiliary volume is integrated into a first stage of the separation upstream of the actual separator.

In a further preferred embodiment of the assembly at least 10%, preferably at least 30%, of a fluid-accessible total volume of the pipe conduit system is formed by the auxiliary volume.

The greater the auxiliary volume compared to the gas-accessible volume of the rest of the pipe conduit system, the more the total gas-accessible volume of the pipe conduit system can be influenced via a fill level alteration of the liquid in the auxiliary volume. It is therefore preferable for at least 10%, in particular at least 30%, of a fluid-accessible total volume of the pipe conduit system to be formed by the auxiliary volume. Especially this quantitative specification distinguishes the described assembly from a conventional assembly where there is no motivation for such a large auxiliary volume.

A fluid may be a gas and/or a liquid. The fluid-accessible volume of the pipe conduit system is thus composed of the gas-accessible volume and the volume occupied by the liquid.

In a further preferred embodiment the assembly further comprises an external storage means connected to the auxiliary volume and at least partially filled with the liquid.

In the present embodiment the fill level of the liquid in the auxiliary volume can be increased at least in a case of a reduction in the volume flow rate of the feed gas stream by introducing the liquid into the auxiliary volume at least partially from the external storage means. The assembly is thus configured such that liquid from the external storage means is introduceable into the auxiliary volume.

The external storage means is preferably connected to the auxiliary volume via a conduit which is distinct from a connecting conduit between the methanol synthesis reactor and the separator.

Especially the external storage means distinguishes the described assembly from a conventional assembly where there is no motivation for such an external storage means.

### BRIEF DESCRIPTION OF THE DRAWINGS

The invention will now be more particularly elucidated with reference to the figures. FIGS. 1 and 3 show a particularly preferred embodiment but the invention is not limited thereto. The figures and the size relationships presented therein are only schematic. In the figures:

FIG. 1 shows an inventive assembly for synthesis of methanol.

FIG. 2 shows a separator known from the prior art.

FIG. 3 shows the separator of the inventive assembly from FIG. 1.

### DETAILED DESCRIPTION OF THE PREFERRED EMBODIMENTS

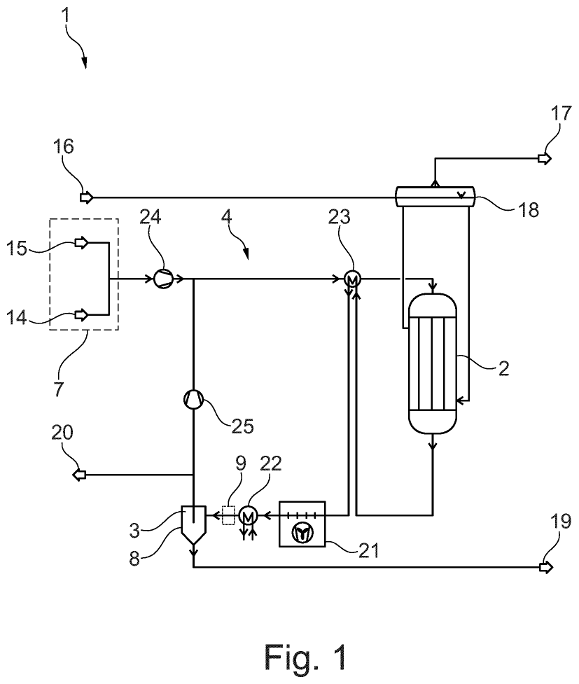

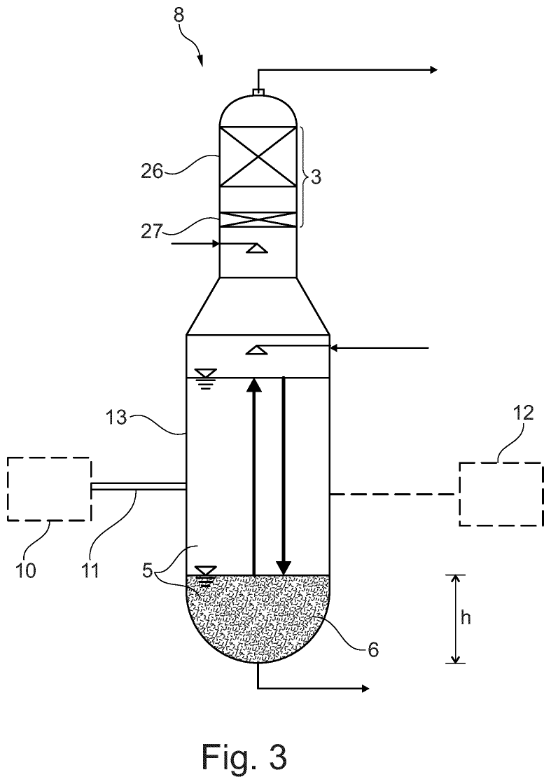

FIG. 1 shows an assembly 1 for synthesis of methanol. The assembly 1 comprises a pipe conduit system 4 and a raw gas source 7, a methanol synthesis reactor 2 and a separation means 3 which are integrated into the pipe conduit system 4. The separation means 3 is configured in a separator 8 as is shown in detail in FIG. 3. The raw gas source 7 comprises an H₂ source 14 and a CO₂ source 15. The raw gas source 7, the methanol synthesis reactor 2 and the separation means 3 in the separator 8 are connected to one another via the pipe conduit system 4. The assembly 1 further comprises a water feed conduit 16 which is connected with a steam drum 18. Steam from the steam drum 18 can be discharged via a steam outlet 17.

Further integrated into the pipe conduit system 4 are a first cooler 21, a second cooler 22, a heat exchanger 23, a first compressor 24 and a second compressor 25. A vessel 9 may optionally be integrated into the pipe conduit system 4 between the methanol synthesis reactor 2 and the separation means 3. This is shown with dashed lines at an exemplary position. The vessel 9 may be configured as a knock-out drum. The methanol formed with the methanol synthesis reactor 2 may be separated with the separation means 3 and discharged at the methanol outlet 19. In the event of overpressure in the pipe conduit system 4 gas may be discharged via an overpressure outlet 20.

The methanol synthesis reactor 2 of assembly 1 can produce methanol at a production rate of up to 5000 tonnes per day.

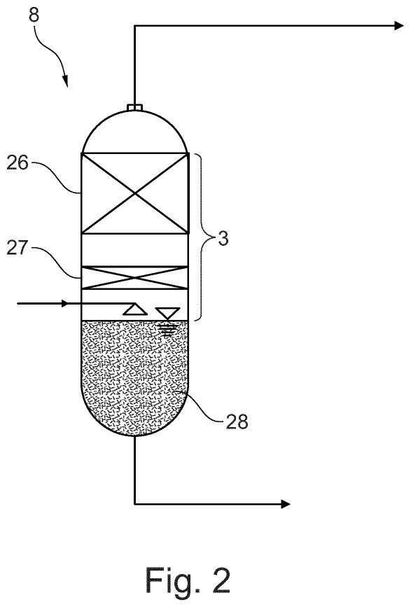

FIG. 2 shows a separator 8 known from the prior art. This comprises a separation means 3 which is formed by a main separator 26 and a pre-separator 27. A liquid region 28 is provided in the lower region of the separator 8.

FIG. 3 shows the separator 8 of the inventive assembly 1 from FIG. 1. This too comprises a separation means 3 which is formed by a main separator 26 and a pre-separator 27.

By means of the separator 8 from FIG. 3 the assembly 1 comprises an auxiliary volume 5 which is integrated into the pipe conduit system 4 such that a gas-accessible volume of the pipe conduit system 4 depends on a fill level h of a liquid 6 in the auxiliary volume 5.

The assembly 1 further comprises a fill level control means 12 which is adapted for increasing the fill level h of the liquid 6 in the auxiliary volume 5 at least in a case of a reduction in a volume flow rate of a feed gas stream supplied to the methanol synthesis reactor 2 from the raw gas source 7 via the pipe conduit system 4. For the sake of clarity the fill level control means 12 is shown only in FIG. 3 and not in FIG. 1.

The separation means 3 and the auxiliary volume 5 are configured in a common housing 13. The auxiliary volume 5 is arranged below the separation means 3.

In contrast to the separator 8 from FIG. 2 the separator 8 from FIG. 3 has a bulbous configuration in its lower region. A cross section of the separator 8 from Fig. is therefore more than twice as large in the region of the auxiliary volume 5 than in the region of the separation means 3. This makes it possible for at least 30% of a fluid-accessible total volume of the pipe conduit system 4 to be formed by the auxiliary volume 5.

The assembly 1 further comprises an external storage means 10 connected to the auxiliary volume 5 via a connecting conduit 11 and at least partially filled with the liquid 6. For the sake of clarity this is shown in FIG. 3 but not in FIG. 1.

### LIST OF REFERENCE NUMERALS

1 Assembly
2 Methanol synthesis reactor
3 Separation means
4 Pipe conduit system
5 Auxiliary volume
6 Liquid
7 Raw gas source
8 Separator
9 Vessel
10 External storage means
11 Connecting conduit
12 Fill level control means
13 Housing
14 H₂ source
15 CO₂ source
16 Water feed conduit
17 Steam outlet
18 Steam drum
19 Methanol outlet
20 Overpressure outlet
21 First cooler
22 Second cooler
23 Heat exchanger
24 First compressor
25 Second compressor
26 Main separator
27 Pre-separator
28 Liquid region
h Fill level

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-13. (canceled)

14. A process for synthesis of methanol comprising:
utilizing a pipe conduit system into which a raw gas source, a methanol synthesis reactor and a separation means are integrated,
supplying a feed gas stream comprising reactants for methanol synthesis from the raw gas source to the methanol synthesis reactor at a volume flow rate,
producing methanol with the reactants in the methanol synthesis reactor,
separating the methanol produced from an outlet stream of the methanol synthesis reactor with the separation means),
wherein the pipe conduit system) further has an integrated auxiliary volume such that a gas-accessible volume of the pipe conduit system depends on a fill level of a liquid in the auxiliary volume and wherein the fill level of the liquid in the auxiliary volume is increased at least in a case of a reduction in the volume flow rate of the feed gas stream.

15. The process according to claim 14, wherein the liquid comprises methanol.

16. The process according to claim 15, wherein the liquid comprises methanol produced in the methanol synthesis reactor.

17. The process according claim 14, wherein the fill level of the liquid in the auxiliary volume is increased at least in a case of a reduction in the volume flow rate of the feed gas stream by introducing the liquid into the auxiliary volume at least partially from an external storage means.

18. The process according to claim 14, wherein the methanol synthesis reactor produces methanol at a production rate of up to 5000 tonnes per day.

19. An assembly for synthesis of methanol comprising:
a pipe conduit system and a raw gas source, a methanol synthesis reactor and a separation mean which are integrated into the pipe conduit system,
the raw gas source, the methanol synthesis reactor and the separation means are connected to one another via the pipe conduit system,
an auxiliary volume which is integrated into the pipe conduit system in such a way that a gas-accessible volume of the pipe conduit system depends on a fill level of a liquid in the auxiliary volume, and
a fill level control means which is adapted for increasing the fill level of the liquid in the auxiliary volume at least in a case of a reduction in a volume flow rate of a feed gas stream supplied to the methanol synthesis reactor from the raw gas source via the pipe conduit system.

20. The assembly according to claim 19, wherein the auxiliary volume is configured in a separator together with the separation means.

21. The assembly according to claim 20, wherein a cross-sectional area of the separator in the region of the auxiliary volume is at least 50% larger than in the region of the separation means.

22. The assembly according to claim 19, wherein the auxiliary volume is arranged below the separation means.

23. The assembly according to claim 19, wherein the auxiliary volume is configured in a vessel which is integrated into the pipe conduit system between the methanol synthesis reactor and the separation means.

24. The assembly according to claim 23, wherein the vessel is configured as a nock-out drum.

25. The assembly according to claim 19, wherein at least 10% of a fluid-accessible total volume of the pipe conduit system is formed by the auxiliary volume.

26. The assembly according to claim 19, further comprising an external storage means connected to the auxiliary volume and at least partially filled with the liquid.
